(19) United States
(12) Patent Application Publication
(10) Pub. No.: US 2016/0270530 A1
(43) Pub. Date: Sep. 22, 2016

(54) Self-Stabilising Four Legged Bases

(71) Applicant: STACKABLE TABLES PTY LTD, Eagle Bay (AU)

(72) Inventors: Christopher Brian Heyring, Eagle Bay (AU); Toby William Heyring, Eagle Bay (AU); Richard Monk, Yalyalup (AU)

(21) Appl. No.: 15/033,907

(22) PCT Filed: Nov. 7, 2014
(86) PCT No.: PCT/AU2014/050342

§ 371 Date: May 3, 2016

(30) Foreign Application Priority Data
Nov. 7, 2013 (AU) 2013904312

(51) Int. Cl. A47B 87/02; F16M 11/22; H04R 1/02; A47B 13/02; A61B 50/10; G09F 7/18; F16M 11/20; A47B 3/08

(52) U.S. Cl. CPC A47B 87/02; F16M 11/2007; F16M 11/22; A47B 3/08; A47B 13/023; A61B 50/10; G09F 7/18; H04R 1/026; A47B 2013/025

(57) ABSTRACT

A stabilising arrangement (base 1) to support an object above four legs (3-6) extending from a hub (2) connected by a stem to the object to be supported. At least one of the legs is pivoted at the hub such that at least two feet (13-16) are adjustable relative to the hub to enable the stabilising arrangement to conform to a warped support surface. Leg geometry of the stabilising arrangement is such that, when a first and a second said stabilising arrangement are horizontally (laterally) stacked with respect to one another, two of the legs of the first or second stabilising arrangements include a region that passes partially under the hub and/or under a region of a leg of the other stabilising arrangement. The first and second legs (3, 4) can be lower than the third and fourth legs (5, 6). Also the first and second legs (3, 4) can be closer (e.g. on legs that are shorter or at 80 degrees) to each other and the third and fourth legs (5, 6) can be wider apart e.g. on legs that are longer or at 100 degrees, to provide a narrower spacing of the feet 13 and 14 that can fit between the feet 15 and 16.

### FIELD OF THE INVENTION

The present invention relates to self-stabilising support stands and bases and is specifically related to four-legged nesting support stands or bases for supporting objects such as signs, speakers, foldable table top and medical equipment.

### BACKGROUND OF THE INVENTION

There are known many different forms of pedestal type base for supporting a variety of objects such as temporary signs, surround sound speakers, medical equipment and foldable table tops (foldable between an operating position in use and a storage position). Some of these pedestal bases have four feet able to conform to uneven ground to provide improved stability with a footprint (the quadrilateral formed by connecting the four ground engaging points or feet) aligned with a square table top for example.

However, when two such self-stabilising bases are stored close together, the legs of one base cannot overlap or pass under or over the legs of a similar base, limiting how closely the bases can be positioned horizontally. This limited stacking density results in the stored bases taking up large amounts of storage space.

There are also known some forms of pedestal type base that are designed to be horizontally stacked with high stacking density, but such bases do not have four legs able to conform to uneven ground surfaces such as non-planar paved surfaces.

### BRIEF DESCRIPTION OF KNOWN ART

Self-stabilising arrangements of pedestal base having four feet are disclosed in U.S. Pat. Nos. 3,814,362, 3,844,517 and 5,690,303, details of each of which are incorporated herein by reference. The pedestal base designs in those documents all provide two orthogonally adjacent feet rigidly fixed to the stem of the support stand, the other two orthogonally adjacent feet being provided on a pivoting beam to enable all four feet to contact a warped ground plane.

Similarly, there are mechanisms in which both pairs of orthogonally adjacent feet are provided on a pair of orthogonally spaced beams, each beam being able to pivot relative to the hub, the rotation of each beam being interrelated by additional parts such as pivoting levers. Examples of this type of mechanism are disclosed in published Australian patent application AU20040100609, international patent publication WO2005/084491, and published Australian patent application AU2012101348.

U.S. Pat. No. 6,209,465 and United States patent application number US2009/183653 both disclose mechanisms in which diagonally opposite pairs of feet are fixed together, so one diagonal beam with two feet is able to move vertically relative to the other diagonal beam with the other two feet.

Arrangements using three or four lever parts are disclosed in international patent application number PCT/AU2010/001745 published as WO2011/075793, details of which are incorporated herein by reference. Each lever part is pivotally connected to a central hub and reacts against at least one adjacent lever part to distribute between the four feet, any warp deformation due to an uneven ground surface.

Similarly, international patent application number PCT/AU2010/001746 published as WO2011/075794, details of which are incorporated herein by reference, discloses arrangements having four lever parts enabling the four feet of the base to conform to uneven surfaces.

There are also hydraulically interconnected arrangements of four feet for pedestal type tables to enable warp compliance of the support arrangement, as disclosed in published Australian patent application AU2008100972 and U.S. Pat. No. 6,009,815.

However none of the above arrangements provide leg geometry suitable for close horizontal stacking.

Conversely, U.S. Pat. No. 6,662,731 discloses a horizontally stackable, four-legged, pedestal style base for a table having a foldable table top, but the four feet and/or legs are rigid so are unable to conform to an uneven ground surface. Two adjacent legs have flattened cross-sections that lie closer to the ground than the opposite two higher or upper legs so that they do not interfere with the higher legs of an adjacent horizontally stacked table. A similar arrangement is disclosed in Chinese utility patent number 201504781 with the feet of the higher legs being narrow to permit the lower legs to slide between them when stacking.

In the present specification, the phrase ‘horizontally stacked’ or the like is not to be taken as limiting to stacking in a precise horizontal plane or direction. ‘Horizontal stacking’ or the like refer to sideways or lateral direction stacking of the base(s) with at least one other such base having compatible stacking features to accommodate such stacking.

It would therefore be desirable to provide self-stabilising base(s) or support stand(s) that permit(s) close sideways/lateral (e.g. horizontal) stacking.

### SUMMARY OF THE INVENTION

With this in mind, according to one aspect of the present invention, there is provided a stabilising arrangement to support an object above four legs extending from a hub, each leg including a respective ground engaging portion; the stabilising arrangement including at least one pivoting part connected to the hub at a respective pivot axis, the or each pivoting part including at least one of the legs and its respective ground engaging portion such that at least two ground engaging portions are adjustable relative to the hub to thereby enable the stabilising arrangement to conform to a warped support surface; wherein a leg portion of a first stabilising arrangement is able to pass at least partially under the hub and/or under a region of at least one leg of a second stabilising arrangement to thereby permit horizontal stacking. For example, the leg portion of the first stabilising arrangement that is able to pass at least partially under the hub and/or under a region of at least one leg of a second stabilising arrangement, may be a stackable profile portion of at least one leg extending from a distal end of the leg towards the hub. The region of the at least one leg that the stackable portion is able to pass under may be a clearance region. The stackable profile portion may have a top surface that is lower than at least a portion of the hub and/or lower than the clearance region.

The respective ground engaging portion may, for example, be selected from one or more of at least one foot, at least one caster, at least one ground engaging point, or other ground engaging means.

The hub of the stabilising arrangement may to be connected by at least one stem to the object to be supported. Preferably the at least one stem is/are arranged with respect to a central axis associated with the hub. Preferably, in the case of a single stem, the stem has a primary axis.

Generally, the stabilising arrangement may include a leg geometry such that, when a first and second said stabilising arrangement are horizontally stacked, a total of two of the legs of the first and second stabilising arrangements (i.e. two of the total of eight legs of the two arrangements) each include a region or leg portion that passes partially under the hub and/or under a region of a leg of the other or the respective other stabilising arrangement. For example: two legs of the first stabilising arrangement may each pass under the hub and/or under a leg of the second stabilising arrangement; or alternatively, one leg of the first stabilising arrangement may pass under the hub and/or under a leg of the second stabilising arrangement and one leg of the second stabilising arrangement may pass under the hub and/or under a leg of the first stabilising arrangement.

For example, each leg may include a top surface extending along a primary portion of the leg, at least two of said four legs may include a region of the top surface that is lower than the hub and/or lower than a region of the under surface of at least two of said four legs, such that when a first and a second of said stabilising arrangements are horizontally stacked, either: a portion of a leg of the first stabilising arrangement passes under the hub and/or a portion of a leg of the second stabilising arrangement and a portion of a leg of the second stabilising arrangement passes under the hub and/or a portion of a leg of the first stabilising arrangement; or a portion of two legs of the first stabilising arrangement passes under the hub and/or a portion of two legs of the second stabilising arrangement. This can thereby enable the distance between the primary axes of the stems of the first and second stabilising arrangements to be minimised. For example, the spacing between the centre axes of the stems of adjacent bases can be less than 200 mm, preferably less than 150 mm or even less than 120 mm. This leg geometry having the pattern of two legs of two adjacent bases passing under an adjacent base to be repeated indefinitely as multiple bases are stacked together efficiently in a row.

Preferably the at least one pivoting part may be rotatably connected to the hub at the respective pivot axis.

The four legs may consist of a first, a second, a third and a fourth leg; the first leg being positioned on the opposite side of the hub to the third leg and the second leg being positioned on the opposite side of the hub to the fourth leg; the first and third legs being substantially parallel to each other and substantially perpendicular to the second and fourth legs.

When a first and second said stabilising arrangement are horizontally stacked, the leg portion of the first stabilising arrangement able to pass at least partially under the hub and/or under a region of at least one leg of the second stabilising arrangement may be portions (such as stackable profile portions) of the first and/or third legs. For example, when a first, second and third stabilising arrangement are horizontally stacked, the first leg of the second stabilising arrangement passes under a portion of the first stabilising arrangement, and the third leg of the second stabilising arrangement passes under a portion of the third stabilising arrangement.

Each leg may have a primary axis, the primary axis of the first and third legs being offset by a width of the first or third leg. For example, when the ground engaging portions lie in a horizontal plane, a vertical plane coinciding with an inner edge of the first leg may be aligned with a vertical plane coinciding with an inner edge of the third leg, or there may be a clearance between the first leg and a vertical plane coinciding with the inner edge of the third leg.

When a first and second said stabilising arrangement are horizontally stacked, the first leg of the second stabilising arrangement may be partially (i.e. for a portion of its length and for at least a portion of its width) under the first leg of the first stabilising arrangement and the third leg of the first stabilising arrangement may be partially (i.e. for a portion of its length and for at least a portion of its width) under the third leg of the second stabilising arrangement. In this case, when a first, second and third said stabilising arrangement are horizontally stacked, the first leg of the second stabilising arrangement may be partially under the first leg of the first stabilising arrangement and partially above the first leg of the third stabilising arrangement and the third leg of the second stabilising arrangement may be partially above the third leg of the first stabilising arrangement and partially under the third leg of the third stabilising arrangement.

Alternatively, when a first and second said stabilising arrangement are horizontally stacked, the respective first or third legs of the first and second stabilising arrangements may be arranged side-by-side. In this case, when a first, second and third said stabilising arrangement are horizontally stacked, the respective first or third legs of the first, second and third stabilising arrangements are arranged side-by-side.

Alternatively, the leg portion of a first stabilising arrangement able to pass at least partially under the hub and/or under a region of at least one leg of the second stabilising arrangement may be a portion (such as a stackable profile portion) of the first and second legs. For example, when a first and second stabilising arrangement are horizontally stacked, the first and second legs of the second stabilising arrangement pass under a portion of the first stabilising arrangement, and the third and fourth legs of the first stabilising arrangement pass over a portion of the second stabilising arrangement. In this case, when a first, second and third stabilising arrangement are horizontally stacked, the first and second legs of the second stabilising arrangement pass under a portion of the first stabilising arrangement, and the third and fourth legs of the second stabilising arrangement pass over a portion of the third stabilising arrangement. When horizontally stacked, the respective first, second, third or fourth legs of the stabilising arrangements may be arranged side-by-side.

The leg portion of each of said four pivoting parts may be angled upwards towards the hub (i.e. downwards away from the hub) in side view. The legs may optionally be angled at a minimum of 10 degrees.

The at least one pivoting part may be a single pivoting part including at least two of said legs and their associated ground engaging portions (such as feet or casters).

Alternatively, the at least one pivoting part may be a first and a second pivoting part, each pivoting part including two of said four legs and their associated ground engaging portions, the stabilising arrangement further including a balance mechanism acting between the first and second pivoting parts, such that rotation of the first pivoting part is opposite (and typically also substantially equal) to rotation of the second pivoting part.

The balance mechanism may include at least one balancing part, the or each at least one balancing part being pivotally connected to the hub and/or stem about a pivot axis perpendicular to the pivot axis of the pivoting parts.

The first pivoting part may include a first and a second leg, the second pivoting part may include a third and a fourth leg, the first leg being opposite the third leg and the second leg being opposite the fourth leg in plan view.

The first and second pivoting parts may each include a lever portion acting on, or being acted on by the balance mechanism. The balance mechanism may include a ring around the stem (i.e. if the lever portions are outside the stem), or partial discs within the stem (i.e. if the lever parts are hidden inside the stem). Preferably the lever portion is substantially vertical.

Alternatively, the least one pivoting part may include a first, second, and third pivoting part: the second pivoting part including first and second engaging regions, the first engaging region being located on the opposite side of the second pivot axis to the second engaging region in plan view; the first pivoting part including a first engaging region, in use engaged with the second engaging region of the second pivoting part, the third pivoting part including a second engaging region, in use engaged with the first engaging region of the second pivoting part, such that rotation of the first pivoting part drives a rotation of the second pivoting part which drives rotation of the third pivoting part in a substantially opposite direction to the first pivoting part to permit a warp displacement of the four ground engaging means.

Alternatively, the at least one pivoting part may include a first, second, third and fourth pivoting part, each having a respective beam portion, the first, second, third and fourth beam portions being arranged to form four sides of a parallelogram in plan view: each respective beam portion including first and second engaging regions, the first engaging region being located on the opposite side of the respective pivoting parts pivot axis in plan view; the first engaging region of the first beam portion in use engaged with the second engaging region of the second beam portion; the first engaging region of the second beam portion in use engaged with the second engaging region of the third beam portion; the first engaging region of the third beam portion in use engaged with the second engaging region of the fourth beam portion; and the first engaging region of the fourth beam portion in use being engaged with the second engaging region of the first beam portion, such that rotation of the first pivoting part drives a rotation of the second pivoting part which drives rotation of the third pivoting part in a substantially opposite direction to the first pivoting part which drives rotation of the fourth pivoting part in a substantially opposite direction to the second pivoting part; each pivoting part including one of said four legs extending from one end of the beam portion.

Each leg may include a ground engaging portion at the distal end of the respective leg, the mechanism thereby permitting a warp displacement of the ground engaging portions. Such warp displacement permits the stabilising arrangement to conform to an uneven ground surface.

For each respective pivoting part, the distance between the respective ground engaging means and the respective pivot axis may be a primary lever rotating moment arm; and the distance between the respective ground engaging means and a portion of the pivot may be a friction loading distance; and the friction loading distance may be greater than or equal to the primary lever-rotating moment arm.

Alternatively, the at least one pivoting part may include a first, second, third and fourth pivoting part, each arranged radially with an inner end towards the hub and an outer end attached to the respective leg, the pivot axes of the four pivoting parts forming a virtual parallelogram in plan view; a protrusion being provided between each respective pair of adjacent pivoting parts, each protrusion being fixed to one pivoting part of said pair of adjacent pivoting parts and extending from that one pivoting part to act on the other pivoting part of said pair of adjacent pivoting parts, such that a support reaction force is transmitted between the pair of adjacent pivoting parts at a point on the other pivoting part between the pivot and the outer end of the beam portion of that other pivoting part, said point being on the opposite side of the pivot axis of the one pivoting part to the outer end of the beam portion of that one pivoting part, such that when in use the outer end, and preferably also the associated leg, of the beam portion of one pivoting part moves in an upwards direction, the outer end, and preferably also the associated leg, of the beam portion of the other pivoting part of the adjacent pair of pivoting parts moves in a downwards direction.

The respective protrusions may include a first protrusion between the first and second leg, a second protrusion between the second and third leg, a third protrusion between the third and fourth leg and a fourth protrusion between the fourth and first leg, each protrusion ensuring substantially opposite vertical motion of the second ends of the beam portions of the associated legs.

The first and second protrusions may extend from the second leg and the third and fourth protrusions extend from the fourth leg, the first protrusion being fixed to the second leg and acting on the first leg, the second protrusion being fixed to the second leg and acting on the third leg, the third protrusion being fixed to the fourth leg and acting on the third leg, and the fourth protrusion being fixed to the fourth leg and acting on the first leg.

Alternatively, the first protrusion may extend from the first leg, the second protrusion extends from the second leg, the third protrusion extends from the third leg and the fourth protrusion extends from the fourth leg.

The object to be supported may be a table top which is hinged to the stem such that it is moveable between an operating (e.g. typically horizontal) position and a stacking (e.g. typically vertical) position.

The or each stabilising arrangement may be provided as a kit of parts or otherwise brought together with the stem and/or the object to be supported (e.g. table top) as a kit of parts.

The invention will be more readily understood from the following description of a number of specific constructions of stabilising arrangements incorporating one or more features of the invention, and as illustrated in the accompanying drawings.

Other arrangements or embodiments are possible, so the provision of the accompanying drawings and the following description thereof should not be taken to limit the scope of the above description of the invention.

### BRIEF DESCRIPTION OF THE DRAWINGS

In the drawings:

FIG. 1 is a perspective view of a first possible base according to the present invention.

FIG. 2 is a plan view of the base shown in FIG. 1.

FIG. 3 is a side view, being a third angle projection from FIG. 2.

FIG. 4 is a perspective view of four horizontally stacked bases of the type shown in FIGS. 1 to 3.

FIG. 5 shows a second possible embodiment of a base according to the present invention.

FIG. 6 is a perspective view of the leg component of the base shown in FIG. 5.

FIG. 7 is a perspective view of four horizontally stacked bases of the type shown in FIG. 5.

FIG. 8 is a plan view of the four horizontally stacked bases of FIG. 7.

FIG. 9 is a perspective view of the four horizontally stacked bases of FIGS. 7 and 8.

FIG. 10 is a perspective view of a third possible base according to the present invention.

FIG. 11 is a plan view of the base shown in FIG. 10.

FIG. 12 is a side view, being a third angle projection from FIG. 11.

FIG. 13 is a perspective view of two horizontally stacked bases of the type shown in FIGS. 10 to 12.

FIG. 14 is a plan view of the two horizontally stacked bases in FIG. 13.

FIG. 15 is a side view, being a third angle projection from FIG. 14.

FIG. 16 is a partially exploded perspective view of a fourth possible base according to the present invention.

FIG. 17 is a perspective view of the leg component of the base shown in FIG. 16.

FIG. 18 is a perspective view of the base shown in FIG. 16.

FIG. 19 is a perspective view of the leg component of the base shown in FIGS. 16 and 18.

FIG. 20 is a perspective view of the base shown in FIGS. 16 and 18.

FIGS. 21 and 22 are perspective views of two horizontally stacked bases of the type shown in FIGS. 16, 18 and 20.

FIG. 23 is an underside view of the two horizontally stacked bases of FIGS. 21 and 22.

FIGS. 24 and 25 are perspective views of a table according to a possible aspect of the present invention.

FIG. 26 is an underside view of the table of FIGS. 24 and 25.

FIG. 27 is a view of the base of the table of FIGS. 24 and 25.

FIG. 28 is a perspective view of three horizontally stacked tables of the type shown in FIGS. 24 to 27.

FIG. 29 is a plan view of the three horizontally stacked tables in FIG. 28.

FIG. 30 is a side view, being a third angle projection from FIG. 29.

FIG. 31 is a side view of a base according to a possible aspect of the present invention.

FIG. 32 is an underside view of the base of FIG. 31.

FIG. 33 is a perspective view of two horizontally stacked bases of the type shown in FIGS. 31 and 32.

### DESCRIPTION OF PREFERRED EMBODIMENT

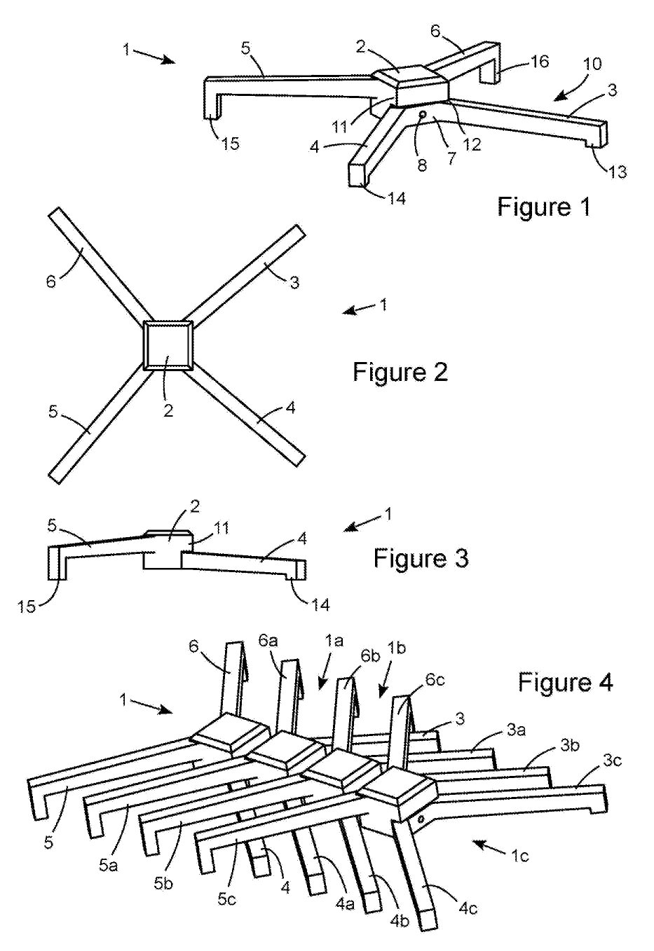

Referring initially to FIG. 1 there is shown a self-stabilising, horizontally stackable base 1 for supporting an object (not shown) via a hub 2. The base 1 has four legs 3, 4, 5, 6 extending from the hub 2, the third leg 5 and the fourth leg 6 being rigidly fixed to or formed as part of the hub 2. The first leg 3 and the second leg 4 are fixed to each other by a beam 7 pivotally connected to the hub by a bolt, rivet, or similar axially retaining shaft or pin 8 such that the legs 3 and 4 are able to rotate together about a pivot axis. This mechanism is similar to mechanisms disclosed in U.S. Pat. Nos. 3,814,362, 3,844,517 and 5,690,303, details of each of which are incorporated herein by reference, and typically has reflectional symmetry.

To prevent the legs spinning upside down or to preferably further limit the rotation of the pivoting leg part 10 (comprising the first leg 3, second leg 4 and beam portion 7) to limit the magnitude of articulation of the mechanism, a limit stop can be provided such as the overhanging portion 11 of the hub 2 above the beam portion 7. For example, while it may be desirable to accommodate a certain magnitude of warp of the ground surface, the mechanism can be limited to motions of for example 10, 15 or even 20 mm of substantially vertical motion of each foot 13 and 14 of the first and second legs (3, 4) relative to the feet 15 and 16 of the fixed legs 5 and 6. This prevents instability due to unnecessarily large motions of the mechanism caused for example by large, eccentrically applied loads.

The term foot is a general term used herein for any form of ground engaging means, such as a ground engaging portion, of the respective leg. Examples may include a fixed rigid or compliant foot, or a wheel such as a caster.

FIG. 1 shows the pivoting leg part 10 rotated until it contacts the underside of the overhanging portion 11 of the hub as indicated at 12.

Referring now also to FIG. 2 and its third angle projection FIG. 3, both showing the self-stabilising stackable base 1 of FIG. 1 in its mid position, i.e. all the ground engaging portions or points (the feet 13, 14, 15, 16) of the legs 3, 4, 5, 6 are on a common linear plane or flat ground surface. The first and second legs (3, 4) are lower than the third and fourth legs (5, 6). Also the first and second legs (3, 4) are closer to each other, for example in FIG. 2 they are not perpendicular, but at 80 degrees to each other and the third and fourth legs (5, 6) are wider apart at 100 degrees to provide a narrower spacing of the feet 13 and 14 that can fit between the feet 15 and 16.

Alternatively or additionally, the length of the legs can be different, such as the first and second legs being shorter than the third and fourth legs which can also help provide a narrower spacing of two of the feet to fit between the other two feet of an adjacent base.

As can be seen in FIG. 4, this permits multiple such bases 1, 1a, 1b, 1c to be closely horizontally stacked, as regions of two (e.g. lower) adjacent legs (in this case the first and second legs 3 and 4, 3a and 4a, 3b and 4b) of a base pass under regions of the other two (e.g. upper) adjacent legs (in this case, the third and fourth legs 5a and 6a, 5b and 6b, 5c and 6c) of an adjacent base. In this case the first and second legs 3 and 4, 3a and 4a, 3b and 4b include stackable profile portions which pass under the regions of the third and fourth legs 5a and 6a, 5b and 6b, 5c and 6c. This permits the bases to be horizontally stacked with a minimal horizontal offset between adjacent bases.

As shown by the horizontal stacking of the four similar bases in FIG. 4, any number of bases can be horizontally stacked together, providing excellent stacking density.

The legs can be spaced perpendicular to each other for a more pleasing aesthetic, and the feet or ground engaging portions 15 and 16 of the upper legs can be shaped to provide a wide spacing therebetween (for example by being elongate in the direction of stacking and being placed as wide apart as possible under the legs 5 and 6) to help minimise the necessary difference in width between the ground engaging portion (e.g. foot) ends of the upper and lower legs.

Preferably, at least a portion of the respective leg(s) to pass at least partially under the hub of a next adjacent base structure can also have a significantly greater width in plan view than thickness in side view to lower the overall height of the base and change the appearance of the base to help minimise the visual difference between the upper legs and the lower legs. More preferably, each of the legs of the associated base structure has the significantly greater width in plan view than thickness in side view. For example, towards the ground engaging portion the bending loads are less than towards the hub so the section of the leg can be thinner towards the ground engaging portion. Having a low leg thickness can also aid stacking within a low overall base profile.

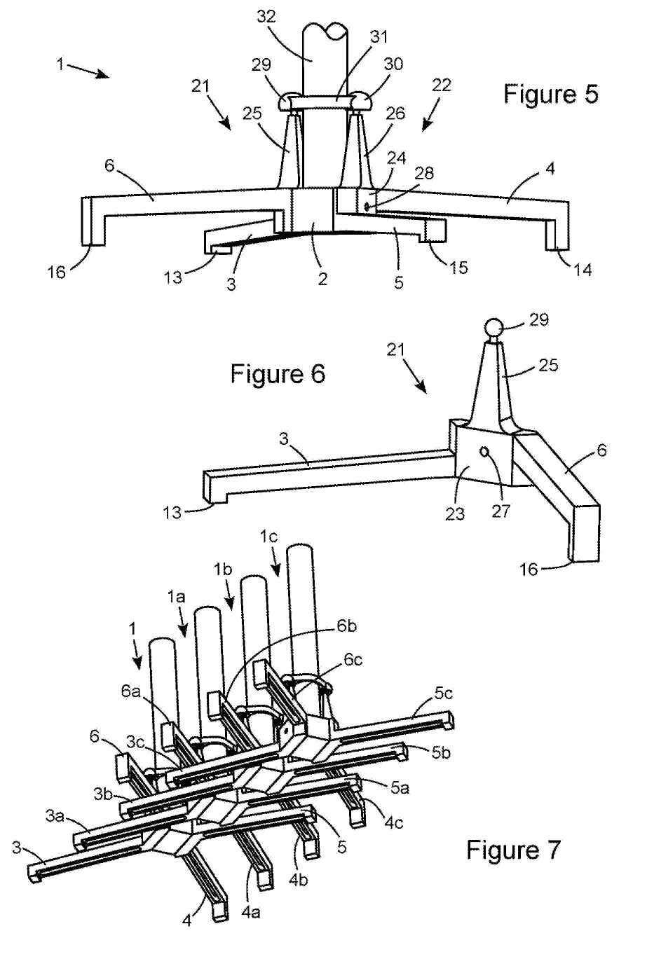

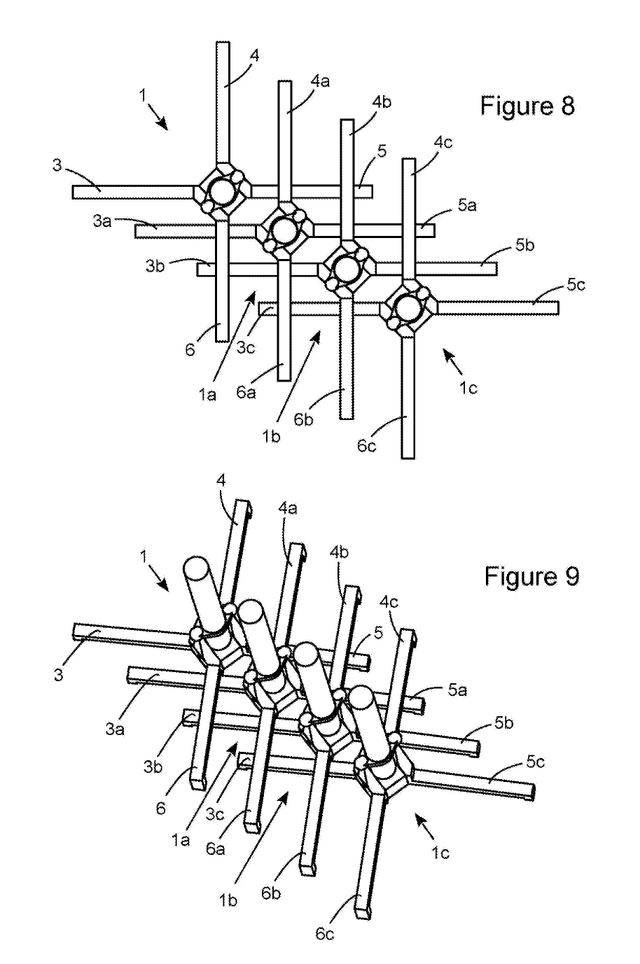

FIGS. 5 to 9 how an alternative form of self-stabilising base mechanism and a different geometry of the four legs enabling stacking. Throughout the drawings, like reference numerals are used for similar or equivalent components.

As shown in FIG. 5, the base 1 no longer has two legs fixed rigidly to the hub 2. Instead two legs (3, 6) are provided on a first pivoting part 21 and the other two legs (4, 5) are provided on a second pivoting part 22. If the upper and lower legs of the base were arranged as in FIGS. 1 to 4 with two orthogonally adjacent legs being lower legs and two orthogonally adjacent legs being upper legs, then the first and second pivoting parts would be different. For example one of the pivoting parts would have two lower legs and the other pivoting part would have two upper legs, or alternatively, although each pivoting part could have an upper and a lower leg, the two pivoting parts would be mirrors of each other (i.e. handed or having reflectional symmetry, not identical). However in this example, the first and second pivoting parts are identical, each having an upper leg and a lower leg (i.e. the base 1 has 2-fold rotational symmetry about the primary axis of the stem).

The first pivoting part 21 is shown in FIG. 6 and includes the first (in this case lower) leg 3 and the fourth (in this case upper) leg 6, joined together by a beam portion 23, to which is also fixed a lever arm 25. The first pivoting part has a pivot axis through the pin, bolt hole, rivet or other suitable fixing 27 and part of a ball joint 29 or similar at the distal end of the lever arm 25.

As can be seen in FIG. 5, the lever arm 25 of the first pivoting part 21 is connected to the lever arm 26 of the second pivoting part 22 by the ball joints 29 and 30 on the balancing ring or lever part 31. The balancing ring 31 is located around the stem 32 which is fixed to or part of the hub 2 of the base 1. As the foot 13 of the first leg 3 moves upwards relative to the hub 2, the first pivoting part rotates about its pivot 27 (only visibly in FIG. 6) moving the foot 16 of the fourth leg 6 downwards relative to the hub 2 and the lever arm 25 swings. This moves the ball in the elongated ball joint 29 and rotates the balancing ring 31 which in turn moves the ball joint 30 and drives lever arm 26 to rotate the second pivoting part about is pivot 28. Therefore the foot 14 of the second leg 4 moves downwards and the foot 15 of the third leg 5 moves upwards, i.e. the mechanism articulates or moves in the warp mode enabling the feet 13, 14, 15, 16 to conform to uneven ground surfaces. This mechanism is similar to mechanisms disclosed in published Australian patent application AU20040100609, WO2005/084491, and AU2012010348, details of which are incorporated herein by reference.

However, as noted above, each pivoting part 21 and 22 has an upper leg and a lower leg, so the base 1 shown in FIG. 5 has two upper legs 4 and 6 that are on opposite sides of the hub and two lower legs 3 and 5 that are similarly opposite each other. These pairs of either upper or lower legs are also shown parallel to each other and substantially in-line with each other.

Therefore the base shown in FIG. 5 can be horizontally, i.e. sideways, stacked as shown in FIGS. 7 to 9. Four bases 1, 1a, 1b, and 1c are shown with regions (for example stackable profile portions) of the lower legs 5, 3a, 5a, 3b, 5b and 3c passing respectively under regions (such as clearance regions) of the upper legs 4a and 4b, 6, 4b and 4c, 6a and 6, 4c and 6b and 6a. The first legs (3, 3a, 3b, 3c) of adjacent bases are adjacent each other side by side and similarly the third legs (5, 5a, 5b, 5c) are arranged side by side in this form of stacking geometry.

As can be seen by the horizontal stacking of the four similar bases in FIGS. 7 to 9, any number of bases can be horizontally stacked together, providing excellent stacking density. If further shaping of the hub and pivoting parts is undertaken, the stacking density can be further improved. For example, taking the first lever part in FIG. 6, part of the beam portion 23 can be removed up to the underside of the upper leg 6 and similarly material can be removed from the hub to permit the lower leg of an adjacent base to be more closely stacked. However, while it is possible to modify the design to enable the bases to be stacked so that the hubs of adjacent bases are almost touching, often the object being supported (such as a sign, book holder or folding table top) will limit the stacking density.

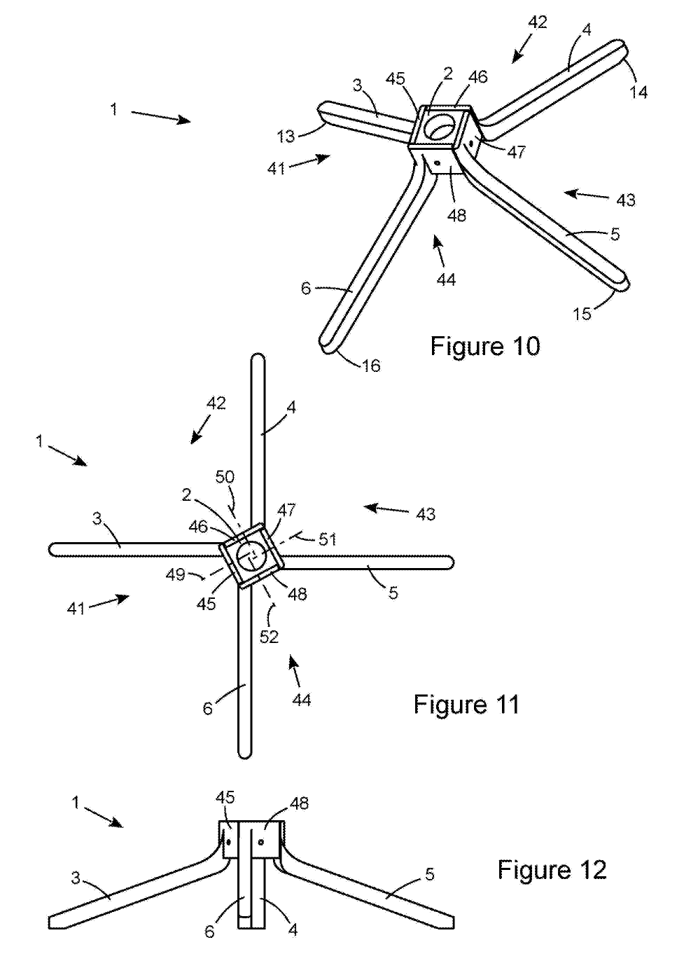

FIG. 10 shows a further alternative form of self-stabilising base mechanism and a further alternative form of geometry of the four legs to enable horizontal stacking. Each of the four legs 3, 4, 5 or 6 is on its own pivoting part 41, 42, 43 or 44 including a respective beam portion 45, 46, 47 or 48 pivotally connected to the hub 2. Adjacent pivoting parts transfer forces between each other through devices such as a pin on one beam portion acting on the edge of a slot in the adjoining beam portion. In this way, a upwards motion of the ground engaging portion 13 of leg 3 rotates the first pivoting part 41 relative to the hub 2. This moves the adjoining edges of the beam portions 45 and 46 of the first and second pivoting parts 41 and 42 upwards, and the accommodating rotation of the second pivoting part moves the second leg 4 and its ground engaging portion 14 downwards. The adjoining edges of the beam portions 46 and 47 of the second and third pivoting parts 42 and 43 therefore move downwards, and the accommodating or resulting rotation of the third pivoting part moves the third leg 5 and its ground engaging portion 15 upwards. The adjoining edges of the beam portions 47 and 48 of the third and fourth pivoting parts 43 and 44 in turn move upwards, and the accommodating rotation of the fourth pivoting part moves the fourth leg 6 and its ground engaging portion 16 downwards. The adjoining edges of the beam portions 48 and 45 therefore move downwards and the mechanism articulates or moves in the warp mode enabling the feet 13, 14, 15, 16 to conform to uneven ground surfaces. This mechanism is similar to mechanisms disclosed in international patent application number PCT/AU2010/001745 published as WO2011/075793, details of which are incorporated herein by reference, as mentioned above.

As can be seen in plan view in FIG. 11, the legs 3, 4, 5 and 6 do not extend from the hub at 45 degrees. The angle between the legs and the pivot axes 49, 50, 51, 52 of the associated pivoting parts 41, 42, 43, and 44 is less than 45 degrees to provide improved stability as disclosed in international patent application number PCT/AU2013/001205 published as WO2014/059481, details of which are incorporated herein by reference. However in this example, even though opposing legs (3 and 5, or 4 and 6) are parallel, they are not in line with other. If each leg is considered to have a primary axis running through the centre of the main straight section of the leg, the primary axis of the first and third legs is offset in plan view by at least a width of the first or third leg. For example, if a vertical plane coinciding with the inner edge of the third leg is extended through the hub to the first leg, that plane will either coincide with the edge of the first leg, or have clearance to the third leg but it will not intersect with the third leg. Each of the legs can be identical as the base 1 has 4-fold rotational symmetry.

The primary axis of each leg is also angled downwards away from the hub as illustrated in the side view FIG. 12.

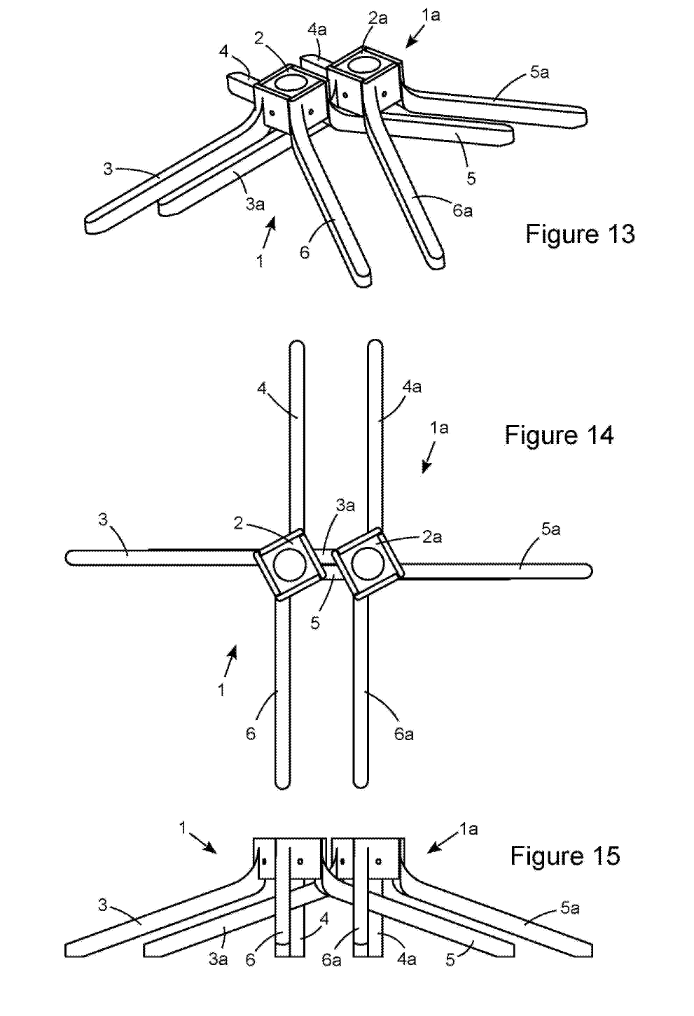

As shown in FIG. 13, this facilitates the first leg 3a (or at least a portion such as a stackable profile portion of the first leg) of an adjacent (i.e. second) horizontally stacked base 1a to pass partially under the first leg 3 of the first base 1. Similarly the third leg 5a of the second base can pass partially over the third leg 5 (i.e. over at least a portion such as the stackable profile portion of the third leg) of the first base 1.

As shown in FIGS. 13 to 15, the first leg 3a of the second base 1a also passes under part of the hub 2 of the first base and similarly the third leg 5 of the first base 1 also passes under a portion of the hub 2a of the second base 1a.

As the plan view FIG. 14 shows, the inside edge of the first leg 3a of the second base 1a is close to and substantially parallel with the inner edge of the third leg 5 of the first base 1 when the bases are closely stacked. However the shaping of the underside of the curved portion of each leg where it is fixed to its associated beam portion determines the clearance between the top of the legs 3a and 5 and the bases 1 and 1a respectively and can be designed to provide latitude in the alignment between the first legs and the first and second bases (and hence between the third legs of the first and second bases). For example the first legs of the adjacent bases need not be totally parallel to each other and the first leg of one base does not need to be in line with and under the first leg of the adjacent base. Adding some magnitude of both of these (the misalignment and the offset between similar legs of adjacent bases) can increase the tolerance when stacking and make the bases easier to stack.

As can be seen from FIGS. 13 and 15, the shaping of the top of each leg (3, 4, 5 or 6) as it joins the associated beam portion (45, 46, 47 or 48) and the downwards angle of the primary axis of the leg, along with the size and shape of the hub and beam portion and the thickness of the legs together determine the minimum horizontal spacing between stacked bases or the maximum stacking density.

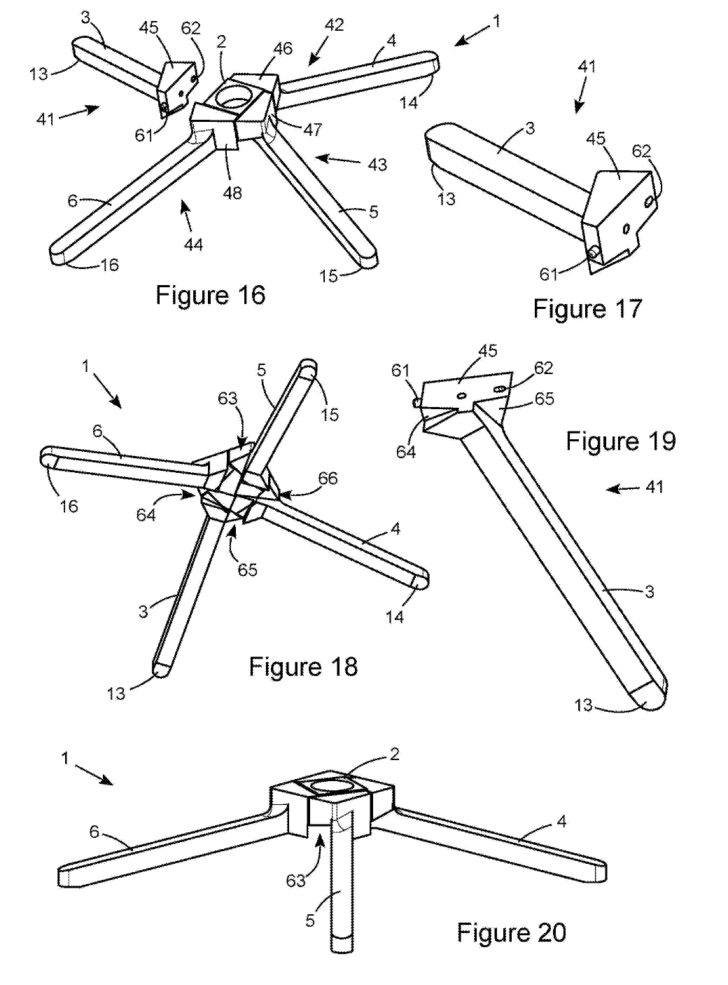

FIGS. 16 to 23 show a similar form of self-stabilising base mechanism to FIG. 10 and a similar form of geometry of the four legs to enable horizontal stacking. As shown in the partially exploded view in FIG. 16, each of the four legs 3, 4, 5 or 6 is again on its own pivoting part 41, 42, 43 or 44 including a respective beam portion 45, 46, 47 or 48 pivotally connected to the hub 2 and adjacent pivoting parts transfer forces between each other at engaging regions on each pivoting part, through devices such as a pin 61 on one beam portion acting on the edge of a slot 62 in the adjoining beam portion. The pivoting part 41 including the first leg 3 is shown in FIG. 17 where the pin 61 and slot 62 can be seen more clearly.

As in FIG. 10, the legs (3, 4, 5, 6) do not extend at 45 degrees to the hub. However in this case, the beam portions 45, 46, 47 and 48 are wedge-shaped in plan view so that the outer faces of the four beam portions form a square in plan view that is aligned with the footprint of the legs (i.e. the quadrilateral having a foot or ground engaging portion of each leg in the four corners). This additional material can increase the minimum horizontal spacing between stacked bases (i.e. reduce the maximum stacking density), so as shown in FIG. 18, four cut-outs 63, 64, 65, and 66 are made through the assembly to allow clearance for a leg of an adjacently stacked base. Each cut-out in this example affects two pivoting parts and the hub. FIG. 20 is a view along the line of one of the cut-outs 63. FIG. 19 shows the first pivoting part 41 with the portions of the two cut-outs that intersect with that pivoting part.

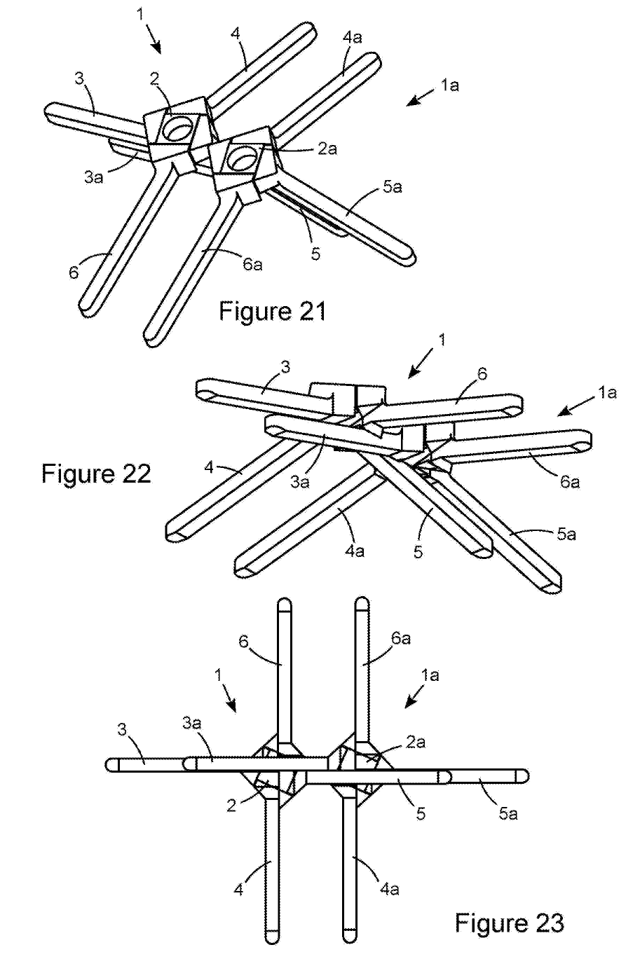

FIGS. 21 to 23 show two bases (of the type shown in FIGS. 16, 18 and 20) horizontally stacked. The limit to how close together the bases can be stacked is now the size of the beam portions.

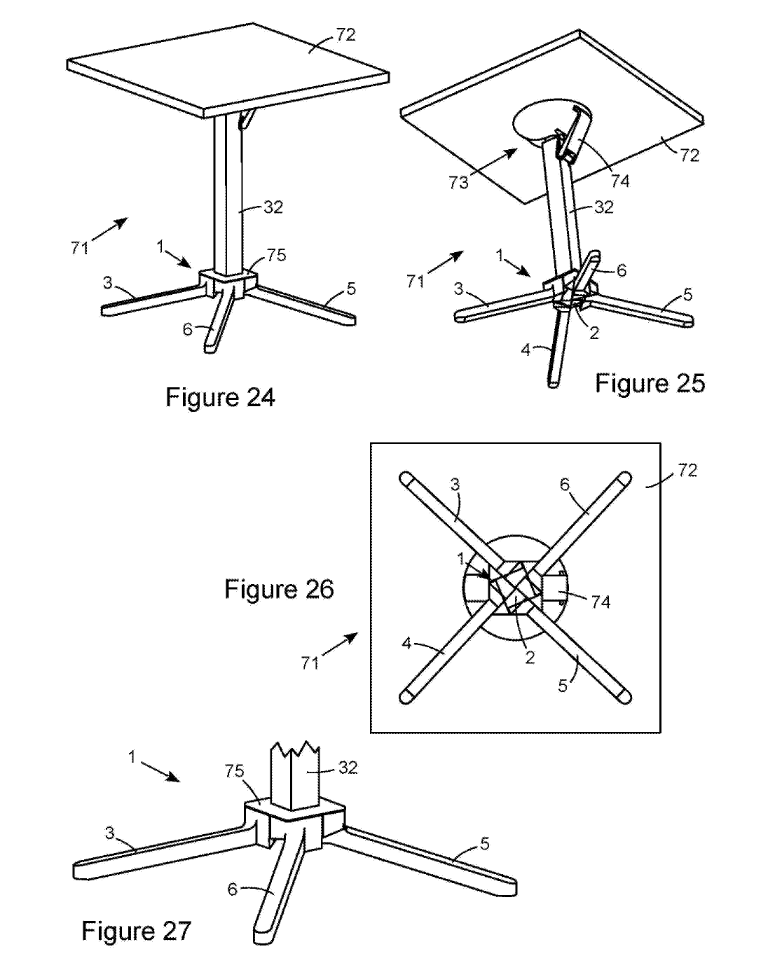

FIGS. 24 to 26 show the same type of base applied to a table 71. The base 1 supports a foldable table top 72 on a square profile stem 32. The folding mechanism 73 includes a latch 74 to lock the table top in the operational position shown or in a stored position. The base, which can be more seen in more detail in FIG. 27, also includes a top plate 75 which performs two functions. Firstly, the top plate provides a limit stop to prevent unnecessarily large motions of the mechanism and secondly it provides a shield or cover over the mechanism to limit the possibility of dirt ingress. The top plate 75 can be flat and sit on top of the hub if the hub top surface is slightly raise from the top surface of the beam portions as shown in FIGS. 16 and 20. Alternatively the top plate can be shaped to provide the required limiting position of the pivoting parts of the mechanism.

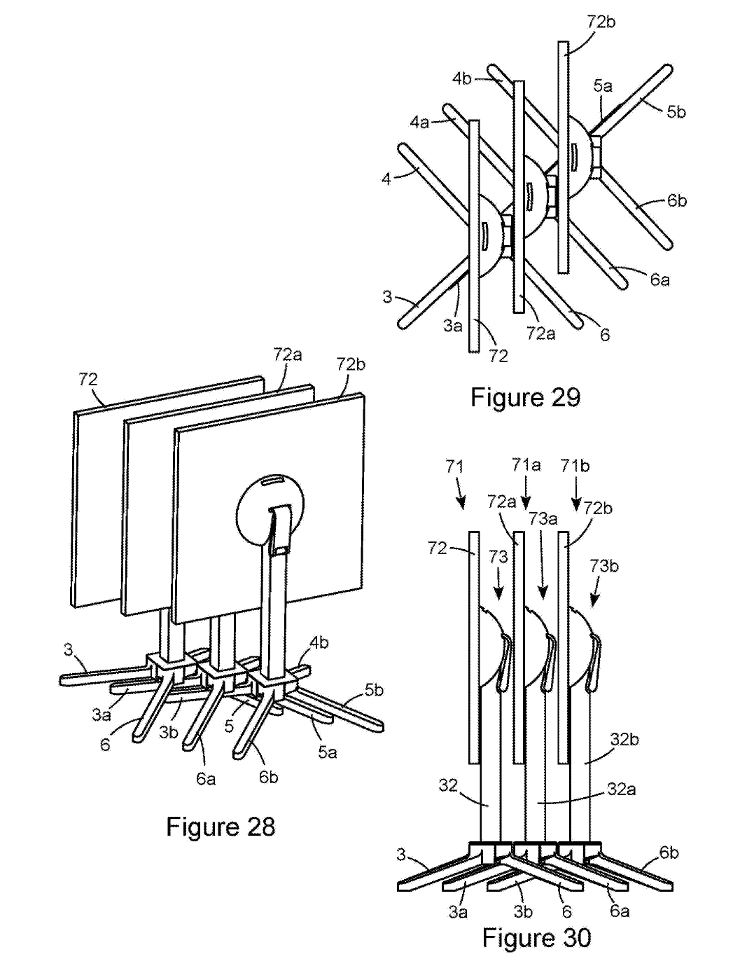

FIGS. 28 to 30 show three such tables 71, 71a and 71b horizontally stacked with the table tops 72, 72a, and 72b in the stored position. In this example, the size of the stem (32, 32a, 32b), table top (72, 72a, 72b) and folding mechanism (73, 73a, 73b) require a larger spacing between the bases (viewed most clearly in FIG. 30) than in FIGS. 21 to 23.

As shown in the illustrated examples, it is possible to combine any form of four-legged self stabilising base with any form of close horizontal stacking leg geometry. For example, the single pivoting beam mechanism of FIG. 1 can be combined with the stacking leg geometry from FIG. 5 where each base has similar opposing leg heights and similar legs of adjacent bases are side by side when stacked.

The mechanism of FIG. 1 can also be combined with the stacking leg geometry of FIG. 10 where similar legs of adjacent bases stack partially under one another, although the legs can extend at closer to 45 degrees to the hub.

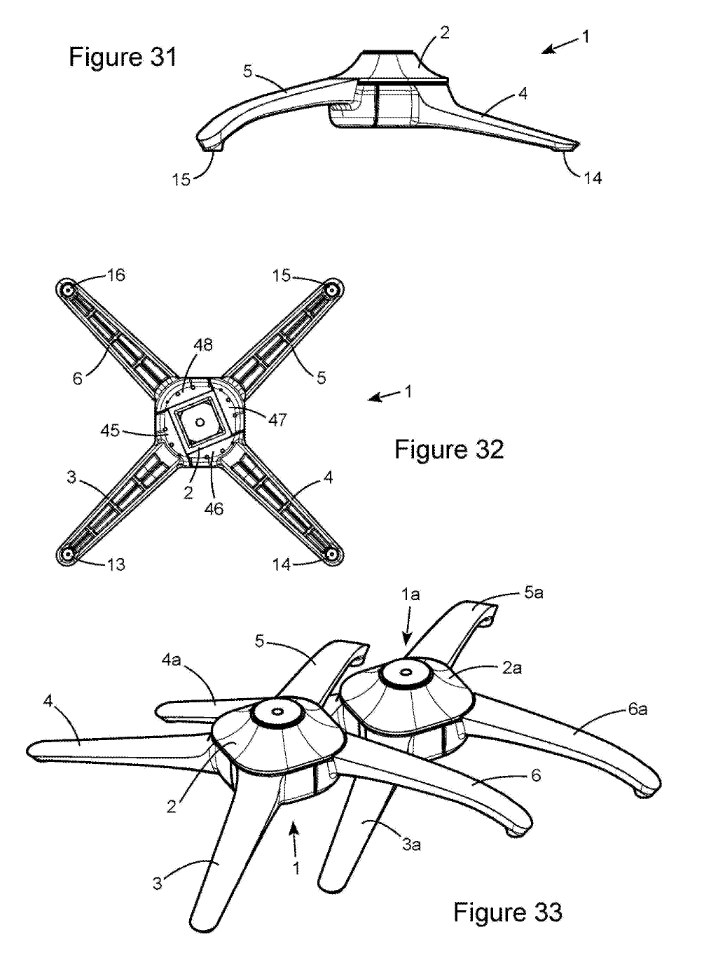

Similarly the mechanisms of FIGS. 10 to 30 can be designed with the leg geometry from FIG. 1 or 5 so similar legs of adjacent stacked bases are side by side and either opposite (say the first and third) legs can slide or pass under adjacent bases, or two adjacent (lower) legs of one base slide or pass under two adjacent (upper) legs of an adjacent base. For example, FIG. 31 shows a side view of a base looking similar to the base of FIG. 3 having two upper legs (leg 5 being visible) and two lower legs (leg 4 being visible), i.e. it has the stacking leg geometry of FIGS. 1 to 4, however the mechanism used is similar to the stabilising mechanism in FIGS. 10 to 30 and the hub 2 is cast to include an integral top plate limit stop.

As can be seen in FIG. 32, the orientation of the footprint of the feet 13-16 is not aligned at 45 degrees to the orientation of the hub 2. Although it is possible to orient the footprint of the feet at 45 degrees to the orientation of the hub, it is preferable to use the orientation shown in FIG. 32 and also in FIGS. 10 to 30 to gain the performance advantages outlined in the aforementioned international patent publication WO2014/059481. However the outer surfaces of the beam portions 45-48 are aligned at 45 degrees to the legs 3-6 as in FIGS. 16 to 30. FIG. 33 shows two such bases 1 and 1a horizontally stacked together.

It is also envisaged that other self-stabilising mechanisms such as additional mechanisms shown in international patent applications PCT/AU2010/001745 published as WO2011/075793 and PCT/AU2010/001746 published as WO2011/075794, details of each of which are incorporated herein by reference, as mentioned above, but not shown in the drawings of the present invention can also be combined with the close stacking leg geometries disclosed herein. Such modifications and variations as would be apparent to the skilled addressee are deemed to be within the scope of the present invention.

1. A stabilising arrangement to support an object above four legs extending from a hub, the hub to be connected by a stem to the object to be supported, each leg including a respective ground engaging portion,
the stabilising arrangement including at least one pivoting part connected to the hub at a respective pivot axis, the or each pivoting part including at least one of the legs and its respective ground engaging portion such that at least two ground engaging portions are adjustable relative to the hub to thereby enable the stabilising arrangement to conform to a warped support surface,
wherein a leg portion of a first stabilising arrangement is configured to pass at least partially under the hub and/or under a region of at least one leg of a second stabilising arrangement to thereby permit horizontal stacking.

2. A stabilising arrangement according to claim 1 wherein the four legs consist of a first, a second, a third and a fourth leg,
the first leg being positioned on the opposite side of the hub to the third leg and the second leg being positioned on the opposite side of the hub to the fourth leg,
the first and third legs being substantially parallel to each other and substantially perpendicular to the second and fourth legs.

3. A stabilising arrangement according to claim 2 wherein the leg portion of the first stabilising arrangement able to pass at least partially under the hub and/or under a region of at least one leg of the second stabilising arrangement is a portion of the first or third leg.

4. A stabilising arrangement according to claim 3 wherein each leg has a primary axis, the primary axis of the first and third legs being offset by a width of the first or third leg.

5. A stabilising arrangement according to claim 3 wherein when horizontally stacked, the first leg of the second stabilising arrangement is partially under the first leg of the first stabilising arrangement and the third leg of the second stabilising arrangement is partially above the third leg of the first stabilising arrangement.

6. A stabilising arrangement according to claim 3 wherein, when horizontally stacked, the respective first or third legs of the first and second stabilising arrangements are arranged side-by-side.

7. A stabilising arrangement according to claim 2 wherein the leg portion of a first stabilising arrangement able to pass at least partially under the hub and/or under a region of a leg of the second stabilising arrangement is a portion of the first leg and a portion of the second leg.

8. A stabilising arrangement according to claim 7 such that when horizontally stacked, the respective first, second, third or fourth legs of the first and second stabilising arrangements are arranged side-by-side.

9. A stabilising arrangement according to claim 1 wherein each of said four legs is angled upwards towards the hub in side view.

10. A stabilising arrangement according to claim 1 wherein the at least one pivoting part is a single pivoting part including at least two of said legs.

11. A stabilising arrangement according to claim 1 wherein the at least one pivoting part is a first and a second pivoting part, each pivoting part including two of said four legs,
further including a balance mechanism acting between the first and second pivoting parts, such that rotation of the first pivoting part is opposite to rotation of the second pivoting part.

12. A stabilising arrangement according to claim 1 wherein the least one pivoting part is a first, second and third pivoting part,
the second pivoting part including first and second engaging regions, the first engaging region being located on the opposite side of the second pivot axis to the second engaging region in plan view,
the first pivoting part including a first engaging region, in use engaged with the second engaging region of the second pivoting part, the third pivoting part including a second engaging region, in use engaged with the first engaging region of the second pivoting part,
such that rotation of the first pivoting part drives a rotation of the second pivoting part which drives rotation of the third pivoting part in a substantially opposite direction to the first pivoting part to permit a warp displacement of the four ground engaging means.

13. A stabilising arrangement according to claim 1 wherein the at least one pivoting part includes a first, second, third and fourth pivoting part, each having a respective beam portion, the first, second, third and fourth beam portions being arranged to form four sides of a parallelogram in plan view,
each respective beam portion including first and second engaging regions, the first engaging region being located on the opposite side of the respective pivoting parts pivot axis in plan view,
the first engaging region of the first beam portion in use engaged with the second engaging region of the second beam portion,
the first engaging region of the second beam portion in use engaged with the second engaging region of the third beam portion,
the first engaging region of the third beam portion in use engaged with the second engaging region of the fourth beam portion, and
the first engaging region of the fourth beam portion in use being engaged with the second engaging region of the first beam portion,
such that rotation of the first pivoting part drives a rotation of the second pivoting part which drives rotation of the third pivoting part in a substantially opposite direction to the first pivoting part which drives rotation of the fourth pivoting part in a substantially opposite direction to the second pivoting part.
each pivoting part including one of said four legs extending from one end of the beam portion.

14. A stabilising arrangement according to claim 13 wherein for each respective pivoting part, the distance between the respective ground engaging means and the respective pivot axis is a primary lever-rotating moment arm, and
the distance between the respective ground engaging means and a portion of the pivot is a friction loading distance, and
wherein the friction loading distance is greater than or equal to the primary lever-rotating moment arm.

15. A stabilising arrangement according to claim 1 wherein the at least one pivoting part includes a first, second, third and fourth pivoting part, each arranged radially with an inner end towards the hub and an outer end attached to the respective leg, the pivot axes of the four pivoting parts forming a virtual parallelogram in plan view,
a protrusion being provided between each respective pair of adjacent pivoting parts, each protrusion being fixed to one pivoting part of said pair of adjacent pivoting parts and extending from that one pivoting part to act on the other pivoting part of said pair of adjacent pivoting parts, such that a support reaction force is transmitted between the pair of adjacent pivoting parts at a point on the other pivoting part between the pivot and the outer end of the beam portion of that other pivoting part, said point being on the opposite side of the pivot axis of the one pivoting part to the outer end of the beam portion of that one pivoting part, such that when in use the outer end of the beam portion of one pivoting part moves in an upwards direction, the outer end of the beam portion of the other pivoting part of the adjacent pair of pivoting parts moves in a downwards direction.

16. A stabilising arrangement according to claim 15 wherein the respective protrusions include a first protrusion between the first and second leg, a second protrusion between the second and third leg, a third protrusion between the third and fourth leg and a fourth protrusion between the fourth and first leg, each protrusion ensuring substantially opposite vertical motion of the second ends of the beam portions of the associated legs.
